(19) United States
(12) Patent Application Publication
(10) Pub. No.: US 2012/0148685 A1
(43) Pub. Date: Jun. 14, 2012

(54) METHODS AND COMPOSITIONS FOR TREATING INSULIN RESISTANCE, DIABETES MELLITUS TYPE 2, METABOLIC SYNDROME AND RELATED DISORDERS

(75) Inventor: Markus Rohner, Mohlin (CH)

(73) Assignee: Engergy4Life AG, Mohlin (CH)

(21) Appl. No.: 13/376,438

(22) PCT Filed: Jun. 10, 2010
(86) PCT No.: PCT/IB10/01398

§ 371 Date: Feb. 29, 2012

Related U.S. Application Data
(60) Provisional application No. 61/185,671, filed on Jun. 10, 2009.

(51) Int. Cl. A61K 31/205; A61K 35/74; A61K 31/455; A61K 36/889; A61K 36/82; A61K 31/202; A61P 3/04; A61P 3/10; A61P 3/00; A61P 9/00; A61P 3/06; A61K 36/185; A61K 36/48; A61K 31/366; A61K 36/53; A61K 31/353; A61K 36/42; A61K 36/258; A61K 36/481

(52) U.S. Cl. 424/727; 514/556; 424/757; 424/780; 514/356; 424/729; 514/560; 424/769; 514/453; 424/745; 514/456; 424/758; 424/728

(57) ABSTRACT

The present invention relates to compositions comprising (i) an activator of transport of fatty acids into the mitochondria; (ii) an activator of fatty acid oxidation; and (iii) an inhibitor of fatty acid biosynthesis, and methods for treating insulin resistance, diabetes mellitus type 2, metabolic syndrome, dyslipidemias, obesity, and/or cardiovascular disorders in a subject.

### THE FIELD OF THE INVENTION

The present invention relates to compositions comprising (i) an activator of transport of fatty acids into the mitochondria; (ii) an activator of fatty acid oxidation; and (iii) an inhibitor of fatty acid biosynthesis and methods for treating insulin resistance, diabetes mellitus type 2, metabolic syndrome, dyslipidemias, obesity, dementia and/or cardiovascular disorders in a subject.

### BACKGROUND OF THE INVENTION

Overfeeding and always available food and/or food intake does lead to overnutrition and to insuline resistant herds and is also observed in men. Permanent overfeeding predispose men for overweight and finally obesity. There is a clear correlation between body mass index and relative fat intake of humans (Pudel & Elrott, 2003, J. Ehrnährungsmed. 2:11-15.) Overweight individuals have an increased daily fat intake of about 20 g or more compared with non overweight individuals (Tucker & Kano, 1992, Am J Clin Nutr 56:616-22). This conveys the ectopic accumulation of lipids in the liver and muscle tissue and leads to overflowing ectopic sites with lipides. Excess fatty acids in the cells tends to develop ectopic fatty acid accumulation in muscle and liver tissues and are today assumed to modulate insuline resistance followed by diabetes mellitus type II. Plasma concentrations of free fatty acids are associated with an increase in fat mass. Elevated levels of free fatty acids can lead to insuline resistance and evidence is growing that β-cell function is impaired through lipotoxicity (Wilding, 2007, Diabetic medicine 24: 934-945). Excessive accumulation of Trigylcerides is related to an increased free fatty acid level under conditions the liver is saturated with glycogen and any additional glucose taken up by hepatocytes is shunted into pathways leading to synthesis of fatty acids. The fat tissue of an insuline resistant individual is getting a net exporting tissue of non esterified fatty acids postprandial. The content of non esterified fatty acids is increased in insuline resistant individuals or patients with diabetes type-2 compared with healthy man (Carpentier, 2008, Diabetes & Metabolism 34: 97-107, Savage et al., 2007, Physiol. Rev. 87: 507-520). Excess fatty acids in serum does change the distribution pattern of the four known molecule types of adiponectin (Untersuchungen über die Bedeutungen von Adiponektin bei der Entstehung von Diabetes Typ-2 Diabetes, Freudenberg, Diplomarbeit 2004, Deutsches Institut für Ernährungsforschnung, Potsdam-Rehbrücke).

Circulation of non esterified fatty acid level is also a marker for risk of sudden death in men (Jouven et al., 2001, Circulation, 104:756-761).

Metabolic syndrome (Syndrome X) and especially Diabetes Mellitus type 2 are promoted by obesity and lack of exercise. Lack of exercise and obesity are especially connected to the increased risk of a future insulin resistance with a possible development of Diabetes Mellitus type 2 or of other clinical pictures of metabolic syndrome X (Ernährungsmedizin and Diätetik, Heinrich Kasper, 10th Edition, Publishers Urban & Fischer, 2004). Type 2 diabetes accounts for 90-95 percent of all diabetes and results from insulin resistance in muscle and impaired function of the pancreatic beta-cells that produce insulin in response to dietary sugar (Taylor, S. I., 1999, Cell, 97: 9-12). The mechanisms for Diabetes Type 2 have undergone vigorous research over the past few years. Primary mechanisms of the past focused on insulin and glucose sensitivity at the cell membrane and the health of pancreatic beta-cells. Recent research has revealed several new complexities.

Originally linked to obesity, leptin is now thought to be involved in hypothalamic energy regulation, stimulation of glucose transport, and the inhibition of insulin secretion by pancreatic beta-cells. Two more recently-discovered hormones are resistin and adiponectin. Resistin has been linked to glucose tolerance and hepatic insulin resistance. Adiponectin is linked to glucose regulation and lipid metabolism. Produced in fat cells, both resistin and adiponectin also apparently mediate PPAR-alpha and -gamma (Peroxisome proliferator receptor).

Enzymes are now also thought to be a critical part of the mechanism for Diabetes Type 2. One enzyme seemingly instrumental to leptin regulation tangential to PPAR-gamma repression is SIRT1 (Silent Information Regulator gene). Sirtuins have been implicated in the diabetic mechanism because of their ability to affect genetic expression related to issues of degenerative glucose and insulin metabolism. SIRT1 appears to balance glucose levels by modulating the PGC-1alpha (Peroxisome proliferator-activated receptor-γ-coactivator) molecule and having an influence to CPT-1 (Carnitine Palmitoyltransferase-1) expression. PGC-1alpha is a transcriptional co-activator of CPT-1. When SIRT1 was inhibited in vivo this increased hypoglycemia, increased glucose and insulin sensitivity as well as increased free fatty acids and cholesterol (Rodgers and Puigserver, 2007, PNAS, 12861-12866). On the other hand increased SIRT1 expression reversed these effects, but only in the presence of PGC-1alpha during fasting. Yet another recently-discovered hormone implicated in both forms of diabetes is amylin. Amylin is co-secreted with insulin and complements insulin's actions together with providing a feedback-response mechanism. Other components complicating these mechanisms include glucocorticoids and circulating lipids. Glucocorticoids are found to stimulated adipogenesis (The biology and regulation of preadipocytes and adipocytes in meat animals. J. Anim. Sci. 2009, 87:1218-1246).

These various mechanisms show that Diabetes Type 2 and insuline resistance as well as related conditions such as metabolic syndrome are extremely complex in the development. Thus there is a need for a comprehensive approach to prevent and/or treat these diseases. The object the invention therefore is to provide compositions and methods for treating or preventing insulin resistance, diabetes mellitus type 2, metabolic syndrome, dyslipidemias, obesity, and/or cardiovascular disorders in a subject which comprise such a comprehensive approach.

This object has been achieved by the composition of the invention comprising (i) an activator of transport of fatty acids into the mitochondria; (ii) an activator of fatty acid oxidation; and (iii) an inhibitor of fatty acid biosynthesis and a method for treating insulin resistance, diabetes mellitus type 2, metabolic syndrome, dyslipidemias, obesity, dementia and/or cardiovascular disorders in a subject, the method comprising administering to the subject a therapeutically effective amount of a composition comprising (i) an activator of transport of fatty acids into the mitochondria; (ii) an activator of fatty acid oxidation; and (iii) an inhibitor of fatty acid biosynthesis. It has been found that the combination of this three effectors efficiently reduce intracellular fatty acids from mammalian cells and/or free fatty acids from blood which will cause prevention or alleviation of insulin resistance, diabetes mellitus type 2, metabolic syndrome, dyslipidemias, obesity, dementia and/or cardiovascular disorders.

### SUMMARY OF THE INVENTION

The present disclosure relates generally to compositions comprising (i) an activator of transport of fatty acids into the mitochondria; (ii) an activator of fatty acid oxidation; and (iii) an inhibitor of fatty acid biosynthesis as well as to methods for their use, including methods for treating insulin resistance, diabetes mellitus type 2, metabolic syndrome, dyslipidemias, obesity, and/or cardiovascular disorders in a subject as well as methods for treating dementia in a subject.

The present disclosure provides a composition comprising (i) an activator of transport of fatty acids into the mitochondria; (ii) an activator of fatty acid oxidation; and (iii) an inhibitor of fatty acid biosynthesis.

The present disclosure provides a pharmaceutical composition comprising the composition comprising (i) an activator of transport of fatty acids into the mitochondria; (ii) an activator of fatty acid oxidation; and (iii) an inhibitor of fatty acid biosynthesis and a pharmaceutically acceptable carrier.

The present disclosure provides a method for treating insulin resistance, diabetes mellitus type 2, metabolic syndrome, dyslipidemias, obesity, and/or cardiovascular disorders in a subject, the method comprising administering to the subject a therapeutically effective amount of a composition comprising (i) an activator of transport of fatty acids into the mitochondria; (ii) an activator of fatty acid oxidation; and (iii) an inhibitor of fatty acid biosynthesis.

The present disclosure provides a method for treating dementia in a subject, the method comprising administering to the subject a therapeutically effective amount of a composition comprising (i) an activator of transport of fatty acids into the mitochondria; (ii) an activator of fatty acid oxidation; and (iii) an inhibitor of fatty acid biosynthesis.

The present disclosure provides a method for lowering cholesterol in blood of a subject, the method comprising administering to the subject a therapeutically effective amount of a composition comprising (i) an activator of transport of fatty acids into the mitochondria; (ii) an activator of fatty acid oxidation; and (iii) an inhibitor of fatty acid biosynthesis.

The present disclosure provides a method of reducing free fatty acids from mammalian cells comprising administering to a mammal a therapeutically effective amount of a composition comprising (i) an activator of transport of fatty acids into the mitochondria; (ii) an activator of fatty acid oxidizing enzymes; and (iii) an inhibitor of fatty acid biosynthesis.

The present disclosure provides a method of reducing free fatty acid accumulation in mammalian tissues comprising administering to a mammal a therapeutically effective amount of a composition comprising (i) an activator of transport of fatty acids into the mitochondria; (ii) an activator of fatty acid oxidizing enzymes; and (iii) an inhibitor of fatty acid biosynthesis.

The present disclosure provides a composition comprising (i) an activator of transport of fatty acids into the mitochondria; (ii) an activator of fatty acid oxidizing enzymes; and (iii) an inhibitor of fatty acid biosynthesis for use as a medicament.

The present disclosure provides a composition comprising (i) an activator of transport of fatty acids into the mitochondria; (ii) an activator of fatty acid oxidizing enzymes; and (iii) an inhibitor of fatty acid biosynthesis for use in a method for treating metabolic syndrome, dyslipidemias, obesity, insulin resistance, diabetes mellitus type 2 and/or cardiovascular disorders in a subject, the method comprising administering to the subject a therapeutically effective amount of said composition.

The present disclosure provides the use of a composition comprising (i) an activator of transport of fatty acids into the mitochondria; (ii) an activator of fatty acid oxidizing enzymes; and (iii) an inhibitor of fatty acid biosynthesis for the manufacture of a medicament for treating metabolic syndrome, dyslipidemias, obesity, insulin resistance, diabetes mellitus type 2 and/or cardiovascular disorders in a subject.

### DETAILED DESCRIPTION OF THE INVENTION

The present disclosure provides compositions comprising (i) an activator of transport of fatty acids into the mitochondria; (ii) an activator of fatty acid oxidation; and (iii) an inhibitor of fatty acid biosynthesis.

An activator of transport of fatty acids into the mitochondria includes an agent that promotes the transport of fatty acids from the cytosol into the mitochondria of a cell. Usually the activator of transport of fatty acids into the mitochondria is L-carnitine, a physiologically acceptable derivative and/or a salt thereof or other active agents that promote transport of fatty acids into the mitochondria like genistein. Preferably, the activator of transport of fatty acids into the mitochondria is L-carnitine, a physiologically acceptable derivative and/or a salt thereof. L-carnitine base ((3R)-hydroxy-4-(trimethylammonium)-butyrat), L-carnitine-L-tartrat, acetyl-L-carnitine, propionyl-L-carnitine, valeroyl-L-carnitine, isovaleroyl-L-carnitine, L-carnitine-magnesium-citrate, L-carnitin-fumarate or other known derivates and/or salts of L-carnitine are used suitably as L-carnitine, a physiologically acceptable derivative and/or salts thereof L-carnitine-L-tartrate, acetyl-L-carnitine and/or L-carnitine base are preferred. Thus L-carnitine, a physiologically acceptable derivative and/or a salt thereof is preferably selected from the group consisting of L-carnitine-L-tartrate, acetyl-L-carnitine and/or L-carnitine base. More preferred is L-carnitine-L-tartrate. Also mixtures of the said L-carnitine and said physiologically acceptable derivative and/or a salt thereof can be used, however only one L-carnitin, a physiologically acceptable derivative and/or a salt thereof is preferably used at a time.

If the composition contains L-carnitine, a physiologically acceptable derivative and/or salt thereof, then this is preferably present in an amount of 10-10000 mg, preferably in an amount of 200-5000 mg, more preferably in an amount of 200-2000 mg, especially in an amount of 300-1000 mg related to the amount of L-carnitine base in the composition.

These amounts as well as the amounts of the other components of the composition of the invention as described below are usually based on a presumed daily dose for a mammal of 70 kg body weight. For a body weight deviating from this value, the quantity is to be appropriately adjusted.

An activator of fatty acid oxidation includes agents which increase the gene expression and/or activity of enzymes or receptors involved in regulating fatty acid oxidation. Fatty acid oxidation may be activated through increased gene expression i.e. increased transcription and/or translation, or activation of fatty acid oxidizing enzymes or receptors involved in regulating fatty acid oxidation. For example, fatty acid oxidation may be achieved through increased transcription, translation, or activation of carnitine palmitoyltransferease (CPT-1), medium chain acyl-CoA-dehydrogenase (MCAD), long chain acyl-CoA dehydrogenase (LCAD) and/or increased transcription, translation, or activation of peroxisome proliferator-activated receptor [alpha] (PPARα) or peroxisome proliferator-activated receptor [delta] (PPARγ). Preferably the activator of fatty acid oxidation is an agent which increases the gene expression and/or activity of PPARα and/or PPARγ, preferably PPARα, in the cells. Agents suitable to activate fatty acid oxidation in this manner may be selected from the group consisting of phytanic acid, adiponectin, an agent which increases the endogenous level of adiponectin in the cells, and an agent which decreases the endogenous level of resistin in the cells, Soya isoflavones among the preferred compound is genistein, lemon polyphenols from lemon peels, phytol contained in fish oil, safflower oil, palm oil, DHA (docosahexaenoic acid), EPA (eicosapentaenoic acid), arachidonic acid, cinnamon, ethanolamide and microorganism favouring butyrate production in the gut as Lactobacillus gasseri. Palm oil contains tocotrienols, which are particularly useful as activator of fatty acid oxidation. Usually extracts of palm oil containing tocotrienols are used. Preferred agents are selected from the group consisting of phytanic acid, adiponectin, an agent which increases the endogenous level of adiponectin in the cells, and an agent which decreases the endogenous level of resistin in the cells. More preferred is an agent which increases the endogenous level of adiponectin in the cells and/or an agent which decreases the endogenous level of resistin in the cells. Equivalently preferred are agents selected from the group consisting of soya isoflavones, palm oil or extracts of palm oil containing tocotrienols, DHA and EPA. More preferred are agents selected from the group consisting of palm oil or extracts of palm oil containing tocotrienols, DHA and EPA. Most preferred are DHA and/or EPA which can be comprised by the composition as agents suitable to activate fatty acid oxidation each individually or in combination. If the composition contains DHA, then this is usually present in an amount of 1-20000 mg, preferably in an amount of 10-5000 mg, more preferably in an amount of 10-1000 mg. If the composition contains EPA, then is usually present in an amount of 1-20000 mg, preferably in an amount of 10-5000 mg, more preferably in an amount of 10-1000 mg. Phytanic acid is usually derived from chlorophyll in plant extracts and is commercially available, for example, from Cayman Chemical, Michigan, US. Amounts which can be used in the composition of the invention comprise an amount of 1-100000 mg. If the composition contains Phytanic acid, then this is preferably present in an amount of 10-10000 mg, preferably in an amount of 200-5000 mg in the composition.

Adiponectin is a protein hormone produced and secreted by adipocytes (fat cells). Adiponectin can be produced using cell culture or microbial technology and could be isolated from cells or from cell broth using chromatography steps and can also be administered in pegylated form (Tanita T. et al., Journal of Immunological Methods, 333, February 2008, 139-146) or is commercially available for analytical purposes, for example, from Bio Vision Incorporation, CA, USA. It influences the body's response to insulin and regulates the metabolism of lipids and glucose. Adiponectin also has anti-inflammatory effects on the cells lining the walls of blood vessels. Adiponectin or its pegylated derivative, preferably its pegylated derivative is used. A desired target plasma level for adiponectin in a human subject is >5 μg/mL. A more desired target plasma level for adiponectin in a human subject is >10 μg/mL. There are commercial sources which produce reagents for assays for adiponectin. These include, but are not limited to, Phoenix Pharmaceuticals (Belmont, Calif.) Panomic (Redwood, Calif.), and Linco Research (St. Louis, Mo.). Amounts which can be used in the composition of the invention comprise an amount of 1-5000 mg.

The agent which increases endogenous level of adiponectin in the cells is usually selected from the group consisting of niacin, pantethine, Radix Astragali, astragaloside II, isoastragaloside I, Galegia officinalis, prebiotics increasing butyrate like non-starch Polysaccharides, or fibers with/or without Magnesium and/or vitamin C, probiotics increasing butyrate, secoisolariciresinol diglucoside, flaxseed lignane, ferulic esters, ferulic amides, polyphenolic grape seed extract, fatty acids as eicosanoid and/or omega-3 fatty acids from fish oil or extracted from krill or algaes or microorganism populations (fermented), o-coumaric acid, rutin, extracts from Bittermelone (Momordica charantia), Teufelskralle (Harpagophytum procumbens), Bockshornklee (Trigonella foenum graecum), Copalchirinde (Cortex Copalchi), Efeuktürbis (Coccinia indica) or Ginseng, resveratrol, green tea extracts or isolated EGCG (epigallocatechingallate) thereof. The preferred agent which increases endogenous level of adiponectin in the cells is selected from the group consisting of niacin, pantethine, Radix Astragali, astragaloside II, isoastragaloside I, Galegia officinalis and flaxseed lignane, more preferred are Radix Astragali and/or Galegia officinalis.

Niacin as referred herein comprises niacin (nicotinic acid, pyridine-3-carboxylic acid) and derivatives such as e.g. nicotinic hexaester of D-glucitol. Niacin and pantethine are commercially available, for example, from Lonza AG, Switzerland (Niacin) or Douglas Laboratories, Pennsylvania, USA (Panthetine). Amounts which can be used in the composition of the invention comprise an amount of 1-10000 mg, preferably from 2 to 10000 mg, more preferably from 2-5000 mg. Radix Astragali is usually provided as an extract of Radix Astragali. Extracts of Radix Astragali feasible for the present invention usually contain astragaloside II and isoastragaloside I and are commercially available, for example, from JoryHerb Ltd., China. Amounts of the extract which can be used in the composition of the invention comprise an amount of 1-50000 mg, preferably present in an amount of 10-5000 mg, more preferably in an amount of 20-4000 mg. Astragaloside II and isoastragaloside I are normally derived from the plant Radix Astragali and are commercially available, for example, from Zhongxin Innova Laboratories, China. Amounts which can be used in the composition of the invention comprise an amount of 1-5000 mg, preferably 1-500 mg. Galega officinalis is usually provided as an extract or as a pharmaceutical substance from Galegin Metformin. Extracts of Galega officinalis feasible for the present invention usually contain Galegin and are commercially available, for example, from Archea-Pharma GmbH Heppenheim (Bergstraβe), Germany. Amounts of the extract which can be used in the composition of the invention comprise an amount of 1-50000 mg. Flaxseed lignane is commercially available, for example, from Natural Ingredient Bio-Resources (Changsha) Co., Ltd, China. Amounts which can be used in the composition of the invention comprise an amount of 1-100000 mg. The agent which decreases the endogenous level of resistin in the cells is selected from the group consisting of glycine, arachidonic acid and eicosapentaenoic acid. Preferred is arachidonic acid or eicosapentaenoic acid or a mixture thereof. Amounts which can be used in the composition of the invention comprise an amount of 1-100000 mg.

An activator of fatty acid oxidation may further comprise agents which increase the AMPK (AMP-activated protein kinase; AMP Adenosinmonophosphate) activity, decrease the ACC (Acetyl-CoënzymeA carboxylase) enzyme activity and/or reduce the allosteric inhibition of malony-CoA (malonyl-CoenzymeA) such as green tea extracts or substances isolated thereof such as EGCG with green tea extracts obtained from Frutarom, Switzerland, applied in concentrations as extract from 0.1 g to 20 g or as isolated EGCG substanz from 1 mg to 10000 mg or other substances such as theaflavine, Bitter melon (Momordia charantia) extract, niacin bound chromium, Chromium Piccolinate or sodium acetic acid. A preferred agent which increases the AMPK activity, decreases the ACC enzyme activity and/or reduces the allosteric inhibition of malony-CoA is green tea extracts or substances isolated thereof such as EGCG. The activator of fatty acid oxidation may further comprise an agent which increases the gene expression and/or activity of fatty acid oxidizing enzymes, in particular the gene expression and/or activity of fatty acid oxidizing enzyme CPT 1. These agents may act via activation of SIRT1. Agents increasing the gene expression and/or the activity of CPT 1 are preferably selected from the group consisting of peptide YY, genistein, daidzein, fenofibrate, tocopherols, conjugated linoleic acid, fructooligosaccharides, sesamin or the extracted compounds sesamolin or episesamin thereof, C75, cerulenin, carbacyclin, Salacia oblonga root extracts, ureido-fibrate 5, fenofibric acid, megestrol acetate, PUFAs (polyunsaturated fatty acids) like DHA and resveratrol. A more preferred agent which increases the gene expression and/or activity of fatty acid oxidizing enzymes is selected from the group consisting of sesamin or the extracted compounds sesamolin or episesamin thereof, genistein, and resveratrol.

Additionally, the composition of the invention may contain an anti-oxidant added to decrease intracellular oxidative damage like allicin extracted from garlic, gingko bilbao, mate tea or mate extract from the leaves of the plant Ilex paraguariensis e.g. as ethanolic extract in powder form such as the commercially available extract EFLA® 920 Green Maté (Frutarom Switzerland Ltd) or glutamine reducing interleukin 6. A preferred anti-oxidant is mate tea or mate extract from the leaves of the plant Ilex paraguariensis.

An inhibitor of fatty acid biosynthesis includes agents which inhibit the de novo fatty acid biosynthesis. De novo fatty acid biosynthesis is regulated, in part, by the LXR nuclear receptor. The LXR nuclear receptor can active numerous transcription factors (such as SREBP-1c), which in turn can activate a number of genes involved in lipogenesis. Therefore, the composition comprises agents that decrease the gene expression and/or activity of enzymes or receptors involved in de novo fatty acid biosynthesis. Agents suitable to inhibit fatty acid biosynthesis in this manner may be selected from the group consisting of cholic acid, chenodeoxycholic acid, oleic acid, C75 (a fatty acid synthase inhibitor), TOFA (5-(Tetradecyloxy)-2-Furoic Acid), FAS, MEDICA, extract of blue algae, Trans10-Cis12-conjugated linoleic acid, tanshinone II from Salvia miltiorrhiza and PUFAs. A further suitable agent to inhibit fatty acid biosynthesis is niacin. The inhibitor of fatty acid biosynthesis of the present invention is preferably selected from the group consisting of cholic acid, chenodeoxycholic acid, oleic acid, C75, TOFA, extract of blue algae, Trans10-Cis12-conjugated linoleic acid, tanshinone II, PUFAs and niacin.

Cholic acid, chenodeoxycholic acid, oleic acid, C75, TOFA, FAS, or MEDICA, are commercially available, for example oleic acid, from Lipoid GmbH Germany, PUFAs (DHA, and others) and niacin are commercially available from Lonza AG, Switzerland. As extract of blue algae preferably an extract of Nostoc commune or an extract of Spirulina species, in particular Spirulina maxima is used. Amounts of the extract used comprise an amount of 1-50000 mg, preferably 100-10000 mg, more preferably 10-5000 mg. Extracts of Nostoc commune and Spirulana species can be obtained, for example, from ALGAEN CORPORATION, NC, USA, or according to the process disclosed in WO2005/074622 or from Nanjing Health Light Business Trade Co., Ltd. Tanshinone II is commercially available, for example, from Dan-shen, Changsha, Hunan, China. Typically tanshinone II is used as tanshinone II obtained as extract from Salvia Miltiorrhiza. Amounts of the extract used comprise an amount of 1-50000 mg, preferably 10-10000 mg.

Typically, amounts which can be used in the composition of the invention comprise an amount of 1-50000 mg cholic acid, 1-50000 mg chenodeoxycholic acid, 1-50000 mg oleic acid, 1-50000 mg TOFA, 1-50000 mg FAS, 1-50000 mg MEDICA, 1-50000 mg extract of blue algae, 1-10000 mg Trans10-Cis12-conjugated linoleic acid, which can be manufactured according US 20090105341, PUFA's (DHA) from 1 to 20000 mg and niacin from 1 to 10000 mg, preferably from 2 to 10000 mg, more preferably from 2-5000 mg.

Preferably the inhibitor of fatty acid biosynthesis is an extract of blue algae or tanshinone II e.g. as extracted from Salvia miltiorrhiza, more preferably an extract of blue algae, most preferably an extract of Nostoc commune. Equally preferably the inhibitor of fatty acid biosynthesis is selected from the group consisting of an extract of blue algae, tanshinone II and niacin, whereas niacin is most preferred.

In some embodiments the composition of the present invention may further comprise an agent which reduces blood glucose i.e. which reduces the level of glucose in the blood, usually measured in whole blood. Subjects at risk of developing type 2 diabetes who demonstrate impaired glucose tolerance are considered to be in a prediabetic state. Glucose tolerance can be measured using glucose challenge tests. There are at least two such tests currently available: the Fasting Plasma Glucose Test (FPG) and the Oral Glucose Tolerance Test (OGTT). In human subjects, a FPG blood glucose level between 100-126 mg/dl of blood is indicative of a prediabetic state and an FPG blood glucose level equal to or greater than 126 mg/dl of blood is indicative of diabetes. An OGTT blood glucose level between 140-199 mg/dl is indicative of prediabetes, and a level equal to or greater than 200 mg/dl is indicative of diabetes. Thus an agent which reduces blood glucose will be able to reduce a blood glucose level of higher than 100 mg/dl to a blood glucose level of lower than 100 mg/dl. Agents which reduce blood glucose can be selected from the group consisting of carnosol or an extract from rosmarine containing carnosol, curcuma or gurmar and water soluble extracts thereof, ceramides, glabridin, licorice flavonoids, phytosterols, pycnogenol extracted from Pinus pinaster, isoleucine, acarbose, extracts from the root and leaves of C. indica, Korea red ginseng roolets, extracts of fenugreek or Mormordica charantia, extracts of Konja-Mannan and American ginseng and Pterocarpus masupium and Sapogenin extracted from Jamaican bitter yam. As well lipoic acid can be used as agent which reduces blood glucose. A preferred agent which reduces blood glucose is carnosol or an extract from rosmarine containing carnosol. Carnosol or an extract from rosmarine containing carnosol are commercially available, for example, from Hunan Carnosol Biotech Ltd., Changsha, Hunan, China, and can be present in the composition in amounts of 5 to 50000 mg.

In some embodiments the composition of the present invention may further comprise an agent which inhibits lipolysis. Lipolysis is a metabolic process in which lipids are broken down and release free fatty acids into the bloodstream. A preferred agent which inhibits lipolysis is niacin or L-arginine. Niacin or L-arginine are commercially available, for example, from Lonza AG, Switzerland (Niacin) and can be present in the composition in amounts of 100 to 10000 mg. If the composition contains L-Arginine, then this is preferably present in an amount of 50-5000 mg, preferably in an amount of 100-1000 mg. If the composition contains niacin, then this is present preferably from 2 to 10000 mg, more preferably from 2-5000 mg.

Each component of the composition of the present invention e.g. the activator of transport of fatty acids into the mitochondria, the activator of fatty acid oxidation, the inhibitor of fatty acid biosynthesis, the anti-oxidans, the agent which reduces blood glucose or the agent which inhibits lipolysis, may comprise a single agent or several different agents having the same mode of action. An activator of fatty acid oxidation may comprise e.g. DHA and EPA which both have the same mode of action i.e. activating fatty acid oxidation.

The activator of transport of fatty acids into the mitochondria; the activator of fatty acid oxidation; and the inhibitor of fatty acid biosynthesis differ from each other i.e. each of them consists of an agent or agents which differ from the agent of the other, i.e. the agent selected as activator of transport of fatty acids into the mitochondria differs from the agent selected as activator of fatty acid oxidation which differs from the agent selected as inhibitor of fatty acid biosynthesis, which differs in turn from the agent selected as activator of transport of fatty acids into the mitochondria. This is an important aspect of the present invention in order to obtain a high synergistic effect of all three components of the composition of the present invention considering that some agents of the composition of the present invention may have several simultaneous modes of actions, e.g. they may activate fatty acid oxidation, thus are an activator of fatty acid oxidation and simultaneously inhibit fatty acid biosynthesis thus are an inhibitor of fatty acid biosynthesis. This is the case e.g. for DHA, which is capable to activate fatty acid oxidation, and is simultaneously capable to inhibit fatty acid biosynthesis and for niacin which is capable to activate fatty acid oxidation, and simultaneously can inhibit fatty acid biosynthesis and additionally can inhibit lipolysis. Thus if the composition comprises DHA e.g. as activator of fatty acid oxidation, another agent such as niacin will be comprised by the composition as inhibitor of fatty acid biosynthesis.

Particular preferred compositions of the present inventions are as follows:

A composition comprising L-carnitine, a physiologically acceptable derivative and/or a salt thereof as activator of transport of fatty acids into the mitochondria; DHA and/or EPA as activator of fatty acid oxidation; niacin as inhibitor of fatty acid biosynthesis.

A composition comprising L-carnitine, a physiologically acceptable derivative and/or a salt thereof as activator of transport of fatty acids into the mitochondria; DHA and/or EPA as activator of fatty acid oxidation; an extract of blue algae as inhibitor of fatty acid biosynthesis. A composition comprising L-carnitine, a physiologically acceptable derivative and/or a salt thereof as activator of transport of fatty acids into the mitochondria; DHA and/or EPA and/or soya isoflavones and/or palm oil or an extract of palm oil containing tocotrienols and/or green tea extracts or substances isolated thereof such as EGCG as activator of fatty acid oxidation; niacin as inhibitor of fatty acid biosynthesis; and L-Arginine as agent which inhibits lipolysis. A composition comprising L-carnitine, a physiologically acceptable derivative and/or a salt thereof as activator of transport of fatty acids into the mitochondria; DHA and/or EPA and soya isoflavones and an extract of palm oil containing tocotrienols as activator of fatty acid oxidation; niacin as inhibitor of fatty acid biosynthesis; and L-Arginine as agent which inhibits lipolysis.

A composition comprising L-carnitine, a physiologically acceptable derivative and/or a salt thereof as activator of transport of fatty acids into the mitochondria; DHA and soya isoflavones and green tea extracts or substances isolated thereof such as EGCG as activator of fatty acid oxidation; niacin as inhibitor of fatty acid biosynthesis; mate tea or mate extract from the leaves of the plant Ilex paraguariensis as anti-oxidans; and L-Arginine as agent which inhibits lipolysis.

A composition comprising L-carnitine, a physiologically acceptable derivative and/or a salt thereof as activator of transport of fatty acids into the mitochondria; DHA and soya isoflavones and green tea extracts or substances isolated thereof such as EGCG and sesamin or the extracted compounds sesamolin or episesamin thereof as activator of fatty acid oxidation; niacin as inhibitor of fatty acid biosynthesis; mate tea or mate extract from the leaves of the plant Ilex paraguariensis and/or garlic as anti-oxidans; an extract from rosmarine containing carnosol as agent which reduces blood glucose and L-Arginine as agent which inhibits lipolysis.

Another object of the invention is a method for treating insulin resistance, diabetes mellitus type 2, metabolic syndrome, dyslipidemias, obesity, and/or cardiovascular disorders, preferably insulin resistance and/or diabetes mellitus type 2 in a subject, the method comprising administering to the subject a therapeutically effective amount of a composition comprising (i) an activator of transport of fatty acids into the mitochondria; (ii) an activator of fatty acid oxidation; and (iii) an inhibitor of fatty acid biosynthesis. The composition used in this method may further comprise an agent which reduces blood glucose and/or an agent which inhibits lipolysis. These kind of agents acts synergistically with the effectors of the basic composition in reducing intracellular fatty acids from cells and/or blood.

Another object of the invention is a method for treating dementia in a subject the method comprising administering to the subject a therapeutically effective amount of a composition comprising (i) an activator of transport of fatty acids into the mitochondria; (ii) an activator of fatty acid oxidation; and (iii) an inhibitor of fatty acid biosynthesis.

Another object of the invention is a method for lowering cholesterol in blood of a subject, preferably in blood of a subject with high cholesterol, the method comprising administering to the subject a therapeutically effective amount of a composition comprising (i) an activator of transport of fatty acids into the mitochondria; (ii) an activator of fatty acid oxidation; and (iii) an inhibitor of fatty acid biosynthesis.

Another object of the invention is the composition described above for use as a medicament. Another object of the invention is the composition described above for use in a method for treating metabolic syndrome, dyslipidemias, obesity, insulin resistance, diabetes mellitus type 2 and/or cardiovascular disorders preferably insulin resistance and/or diabetes mellitus type 2 in a subject, the method comprising administering to the subject a therapeutically effective amount of said composition.

Another object of the invention is the composition described above for treating dementia in a subject, the method comprising administering to the subject a therapeutically effective amount of said composition.

Another object of the invention is the composition described above for lowering cholesterol in blood of a subject, preferably in blood of a subject with high cholesterol, the method comprising administering to the subject a therapeutically effective amount of said composition.

Another object of the invention is the use of the composition described above for the manufacture of a medicament for treating metabolic syndrome, dyslipidemias, obesity, insulin resistance, diabetes mellitus type 2 and/or cardiovascular disorders preferably insulin resistance and/or diabetes mellitus type 2 in a subject.

The methods provided herein embrace treatment methods. As used herein, the term “treatment” refers to the administration of one or more therapeutic agent to a subject for the purpose of achieving a medically desirable benefit. Accordingly, “treatment” intends to embrace both “prophylactic” and “therapeutic” treatment methods. Prophylactic treatment methods refer to treatment administered to a subject at risk of developing a condition. For example, a prophylactic treatment for type 2 diabetes refers to treatment of a subject at risk of developing type 2 diabetes (e.g., a prediabetic subject). Therapeutic treatment usually refer to treatment of a subject after the diagnosis of such a condition. The treatment of dementia as described herein is preferably a prophylactic treatment reducing atherosclerosis.

A subject shall mean a human or animal including but not limited to a dog, cat, horse, pony, cow, pig, sheep, goat, chicken, rodent e.g., rats and mice, primate, e.g., monkey, and fish or aquaculture species such as fin fish (e.g., salmon) and shellfish (e.g., shrimp and scallops), provided that it would benefit from the methods provided herein. Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. In all embodiments human subjects are preferred. Mammals include humans, which are preferred. One category of subjects to be treated according to the invention are those having or at risk of developing type 2 diabetes. Risk factors for type 2 diabetes include obesity, family history of diabetes, prior history of gestational diabetes, impaired glucose tolerance (as discussed above), physical inactivity, and race/ethnicity or genetic predisposition. Symptoms associated with diabetes include but are not limited to frequent urination, excessive thirst, extreme hunger, unusual weight loss, increased fatigue, irritability and blurred vision.

Diabetes is associated with other conditions, many of which result from a diabetic state. These include acute metabolic complications such as diabetic ketoacidosis and hyperosmolar coma, and late complications such as circulatory abnormalities, retinopathy, nephropathy, neuropathy and foot ulcers. A more detailed description of the foregoing terms can be obtained from a number of sources known in the art (see, e.g., Harrison's Principles of Internal Medicine, 15th Edition, McGraw-Hill, Inc., N.Y.). Thus, the methods of the invention also embrace ameliorating or resolving diabetes-associated conditions such as but not limited to those recited above.

Subjects at risk of developing diabetes also may be overweight to the point of being obese. Thus another category of subjects to be treated according to the invention are subjects in the state of being obese. The state of being overweight or obese is defined in terms of the medically recognized body mass index (BMI). BMI equal to a person's body weight (kg) divided by the square of his or her height in meters. A subject having a BMI of 25 to 29.9 is considered overweight. A subject having a BMI of 30 or more is considered obese.

Another category of subjects to be treated according to the invention are subjects with insulin resistance (IR). IR is the condition in which normal amounts of insulin are inadequate to produce a normal insulin response from fat, muscle and liver cells. Insulin resistance in fat cells reduces the effects of insulin and results in elevated hydrolysis of stored triglycerides in the absence of measures which either increase insulin sensitivity or which provide additional insulin. Increased mobilization of stored lipids in these cells elevates free fatty acids in the blood plasma. Insulin resistance in muscle cells reduces glucose uptake (and so local storage of glucose as glycogen), whereas insulin resistance in liver cells results in impaired glycogen synthesis and a failure to suppress glucose production. Elevated blood fatty acid levels (associated with insulin resistance and diabetes mellitus Type 2), reduced muscle glucose uptake, and increased liver glucose production all contribute to elevated blood glucose levels. High plasma levels of insulin and glucose due to insulin resistance are believed to be the origin of metabolic syndrome and type 2 diabetes, including its complications.

Another category of subjects to be treated according to the invention are subjects with metabolic syndrome. Metabolic syndrome (also referred to as syndrome X) is a cluster of risk factors that is responsible for increased cardiovascular morbidity and mortality. The National Cholesterol Education Program-Adult Treatment panel (NECP-ATP III) identified metabolic syndrome as an independent risk factor for cardiovascular disease. (National Institutes of Health: Third Report of the National Cholesterol Education Program Expert Panel on Detection, Evaluation, and Treatment of High Blood Cholesterol in Adults (Adult Treatment Panel III). Executive publication no. 01-3670). As used herein, metabolic syndrome is defined according to the World Health Organization criteria (1999) which require presence of diabetes mellitus, impaired glucose tolerance, impaired fasting glucose or insulin resistance, AND two of the following:

blood pressure: ≧140/90 mmHg
dyslipidaemia: triglycerides (TG): ≧1.695 mmol/L and high-density lipoprotein cholesterol (HDL-C)≦0.9 mmol/L (male), ≦1.0 mmol/L (female)
central obesity: waist:hip ratio >0.90 (male); >0.85 (female), and/or body mass index >30 kg/m2
microalbuminuria: urinary albumin excretion ratio ≧20 mg/min or albumin:creatinine ratio ≧30 mg/g.

Another category of subjects to be treated according to the invention are subjects with cardiovascular disorders. “Cardiovascular disorder”, as used herein, includes elevated blood pressure (hypertension), atherosclerosis, heart failure or a cardiovascular event such as acute coronary syndrome, myocardial infarction, myocardial ischemia, chronic stable angina pectoris, unstable angina pectoris, angioplasty, stroke, sudden death, transient ischemic attack, claudication(s), or vascular occlusion(s).

Risk factors for a cardiovascular disorder include dyslipidemia, obesity, diabetes mellitus, pre-hypertension, elevated level(s) of a marker of systemic inflammation, age, a family history of cardiovascular disorders, and cigarette smoking. The degree of risk of a cardiovascular disorder or a cardiovascular event depends on the multitude and the severity or the magnitude of the risk factors demonstrated by the subject. Risk charts and prediction algorithms are available for assessing the risk of cardiovascular disorders and cardiovascular events in a human subject based on the presence and severity of risk factors.

Another category of subjects to be treated according to the invention are subjects with dyslipidemias. As used herein, dyslipidemia is an abnormal serum, plasma, or blood lipid profile in a subject. An abnormal lipid profile may be characterized by total cholesterol, low density lipoprotein (LDL)-cholesterol, triglyceride, apolipoprotein (apo)-B or Lp(a) levels above the 90th percentile for the general population or high density lipoprotein (HDL)-cholesterol or apo A-I levels below the 10th percentile for the general population. Dyslipidemia can include hypercholesterolemia and/or hypertriglyceridemia. Hypercholesterolemic human subjects and hypertriglyceridemic human subjects are associated with increased incidence of cardiovascular disorders. A hypercholesterolemic human subject is one who fits the current criteria established for a hypercholesterolemic human subject. A hypercholesterolemic subject has an LDL cholesterol level of >160 mg/dL, or >130 mg/dL and at least two risk factors selected from the group consisting of male gender, family history of premature coronary heart disease, cigarette smoking, hypertension, low HDL (<35 mg/dL), diabetes mellitus, hyperinsulinemia, abdominal obesity, high lipoprotein, and personal history of a cardiovascular event, malnutrition and/or a genetic predisposition. A hypertriglyceridemic human subject is one who fits the current criteria established for a hypertriglyceridemic subject. A hypertriglyceridemic human subject has a triglyceride (TG) level of >200 mg/dL.

Dyslipidemias encompassed by this invention include dyslipidemias caused by single gene defects, dyslipidemias that are multifactorial or polygenic in origin, as well as dyslipidemias that are secondary to other disease states or secondary to pharmacological agents. Examples of genetic dyslipidemias include Familial Hypercholesterolemia, Familial Defective Apo BIOO, Familial Hypertriglyceridemia, Familial Apoprotein CII deficiency, Hepatic Lipase Deficiency, Familial Combined Hyperlipidemia, Dysbetalipoproteinemia, and Familial Lipoprotein Lipase Deficiency. One example of multifactorial or polygenic dyslipidemia is Polygenic hypercholesterolemia.

Subjects at risk of developing a dyslipidemia are also encompassed by this invention. Such subjects include subjects with Familial Hypertriglyceridemia, Familial Apoprotein CII deficiency, Hepatic Lipase Deficiency, Familial Combined Hyperlipidemia, Dysbetalipoproteinemia, and Familial Lipoprotein Lipase Deficiency. Subjects having or at risk of developing a dyslipidemia also include subjects who suffer from alcohol abuse or dependence, pancreatitis, glucose-6-phosphatase deficiency, hepatitis, Systemic Lupus Erythematosus (SLE), monoclonal gammopathies (such as, for example, multiple myeloma and lymphomas), and Acquired Immunodeficiency Syndrome (AIDS).

Another category of subjects to be treated by the method for lowering cholesterol in blood of a subject according to the invention are usually subjects with high cholesterol in blood (hypercholesterolemic subjects) such as obese subjects, subjects with metabolic syndrome, subjects with insuline resistance, subjects with diabetes mellitus type II or subjects with dyslipidemia. Usually plasma cholesterol, and/or VLDL/LDL cholesterol and/or HDL cholesterol, in particular HDL cholesterol are lowered at least 10%, preferably 15%, more preferably 20% compared to the untreated subject with high cholesterol in blood by the method of the present invention. A subject with high cholesterol as defined herein refers to a subject having an LDL cholesterol level of >160 mg/dL, or of at least >130 mg/dL.

Subjects with elevated blood pressure (hypertension) as well as subjects with pre-hypertension are also encompassed by the invention. Elevated blood pressure or hypertension is defined as a systolic blood pressure >120 mm Hg, or a diastolic pressure >80 mm Hg or an elevation of both (i.e., systolic blood pressure >120 mm Hg and a diastolic pressure >80 mm Hg). Pre-hypertension is defined as systolic blood pressure between 115 and 120 mm Hg, and/or a diastolic pressure between 75 and 80 mm Hg.

Subjects with dementia are also encompassed by the invention. Dementia is defined as a serious loss of cognitive ability in a previously unimpaired person, beyond what might be expected from normal aging. It may be static, the result of a unique global brain injury, or progressive, resulting in long-term decline due to damage or disease in the body. Although dementia is far more common in the geriatric population, it may occur in any stage of adulthood. Dementia is a non-specific illness syndrome (set of signs and symptoms) in which affected areas of cognition may be memory, attention, language, and problem solving. It is normally required to be present for at least 6 months to be diagnosed.

The compositions of the invention are administered in effective amounts and/or therapeutically effective amounts. They can be applied as nutraceutical composition or, usually in higher doses, as pharmaceutical composition. An effective amount is a dosage of the agent sufficient to provide a neutraceutically desirable result. A therapeutically effective amount is a dosage of the therapeutic agent sufficient to provide a medically desirable result. The therapeutically effective amount may vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. The dosage may be adjusted by the individual physician in the event of any complication.

A effective amount of one of the agents comprised in the composition of the invention applied as nutraceutical composition typically will vary from about 0.01 mg/kg to about 100 mg/kg, from about 0.1 mg/kg to about 75 mg/kg, from about 1 mg/kg to about 50 mg/kg in one or more dose administrations daily, for one or several days (depending of course of the mode of administration and the factors discussed above).

A therapeutically effective amount of one of the agents comprised in the composition of the invention typically will vary from about 0.01 mg/kg to about 1000 mg/kg, from about 0.1 mg/kg to about 750 mg/kg, from about 1 mg/kg to about 500 mg/kg in one or more dose administrations daily, for one or several days (depending of course of the mode of administration and the factors discussed above). Other suitable dose ranges include 1 mg to 100000 mg per day, 10 mg to 10000 mg per day, 50 mg to 1000 mg per day.

Single or multiple doses of the composition are contemplated. Desired time intervals for delivery of multiple doses can be determined by one of ordinary skill in the art employing no more than routine experimentation. Preferably, the composition is administered once a day in order to facilitate patient compliance. Individual agent(s) comprised in the composition can be administered at different times per day e.g. morning, noon, and evening so that at the end of each day the whole composition has been administered as a daily dose. Such way of administration is usually contemplated by using a kit (e.g., a packaged combination of agents in predetermined amounts with instructions) comprising the composition in several packages each of them containing individual agent(s) comprised in the composition and instructions for use. Thus a further aspect of the present invention is a kit comprising the composition of the present invention for the treatment of insulin resistance, diabetes mellitus type 2, metabolic syndrome, dyslipidemias, obesity, dementia and/or cardiovascular disorders.

Preferably, the composition is designed to be delivered with greatest ease to subjects. This may include for example a once a day oral administration, the timing of which is not dependent upon food intake. Thus, for example, the composition can be taken every morning and/or every evening, regardless of when the subject has eaten or will eat.

A variety of administration routes are available. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular or infusion. Intravenous or intramuscular routes are not particularly suitable for long-term therapy and prophylaxis. They could, however, be preferred in emergency situations. Oral administration is a generally preferred mode of administration because of the convenience to the patient.

When used in vivo, the agents are formulated as nutraceutical compositions or preparations or as pharmaceutical compositions or preparations. Thus a further embodiment of the present invention is a nutraceutical composition.

A further embodiment of the present invention is a pharmaceutical composition comprising the composition described above and a pharmaceutically acceptable carrier. A pharmaceutical preparation is a composition suitable for administration to a subject. Such preparations are usually sterile and prepared according to GMP standards, particularly if they are to be used in human subjects. In general, a composition or preparation comprises the agent(s) and a pharmaceutically-acceptable carrier, wherein the agent(s) are generally provided in therapeutically effective amounts. As used herein, a pharmaceutically-acceptable carrier means a non-toxic material that does not interfere with the effectiveness of the biological activity of the agents of the invention.

Pharmaceutically-acceptable carriers include diluents, fillers, salts, buffers, stabilizers, solubilizers and other materials which are well-known in the art. Such preparations may routinely contain salt, buffering agents, preservatives, compatible carriers, and optionally other therapeutic or prophylactic agents. When used in medicine, the salts should be pharmaceutically acceptable, but non-pharmaceutically-acceptable salts may conveniently be used to prepare pharmaceutically-acceptable salts thereof and are not excluded from the scope of the invention. Such pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compositions of the invention may be formulated into preparations in solid, semisolid, liquid or gaseous forms such as tablets, capsules, powders, granules, ointments, solutions, depositories, inhalants and injections, and usual ways for oral, parenteral or surgical administration. For oral administration, the agents can be formulated readily by combining the active compound(s) with pharmaceutically-acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers. Depending on the agents comprised by the composition, a particular useful form for oral administration comprises the agents in powder form and additionally sugars or sugar substitutes and flavours which can easily be homogenized and filled in sachets. Such an administration form is contemplated for a composition comprising e.g. L-carnitine-tartrate, DHA and niacin but not limited to these three compounds. Dragee cores are provided with suitable coatings. For this purpose, solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

A further object of the invention is a method of reducing free fatty acids from mammalian cells comprising administering to a mammal a therapeutically effective amount of a composition comprising (i) an activator of transport of fatty acids into the mitochondria; (ii) an activator of fatty acid oxidizing enzymes; and (iii) an inhibitor of fatty acid biosynthesis. Usually, the mammalian cells are human muscle cells and/or human liver cells.

A further object of the invention is a method of reducing free fatty acid accumulation in mammalian tissues comprising administering to a mammal a therapeutically effective amount of a composition comprising (i) an activator of transport of fatty acids into the mitochondria; (ii) an activator of fatty acid oxidizing enzymes; and (iii) an inhibitor of fatty acid biosynthesis. Preferred mammalian tissues are muscle and/or liver tissues.

The composition used in these methods may further comprise an agent which reduces blood glucose and/or an agent which inhibits lipolysis.

The present invention is explained in more detail with the help of the following examples, without being limited to it.

### EXAMPLES

### Example 1

### Preparation of the Composition

18 g of the components L-carnitine-L-tartrat (LONZA AG, CH-4002 Basel), maltodextrine, natriumcyclamate, natriumcitrate, natriumsaccharine, citronic acid (all from Fluka AG. p. a.), natural lemon flower (Zitronen Aroma 74912-71: Givaudan, CH-8600 Dübendorf), 10 g of extract of Radix astragalus (JoryHerb.Lt, China) and 10 g extract of Nostoc commune (ALGAEN CORPORATION, NC, USA) are mixed until the mixture is homogenous. Optionally other aroma compounds can be added to the mixture. The mixture is either filled in sachets or filled into capsules or other final galenic forms for oral application. The mixture is packed and is portioned for an amount of L-carnitine related to L-carnitine Base of about 300 to 1500 mg/Sachets.

### Example 2

### Decrease of Free Fatty Acid in Human in a Hypothetical Case

A composition according to example 1 is orally applied daily to a hypothetical man diagnosed with insulin resistance having a high level of free fatty acids in plasma typically of about >>100 μmol/l. The composition is orally applied for a time period of 200 days or longer as needed according to patient's status. Each of the components of the composition will contribute to the lowering of the level of free fatty acid as follows: The transport of free fatty acids will be increased of approx. 1-10% by L-carnitine. The Extract of Radix Astragalus will be expected to increase the fatty acid oxidation of about 1-10%. The extract of Nostoc commune will reduce the expression of SREB1 responsive genes such as the fatty acid synthase thus reducing the de novo synthesis of fatty acids by approx 1-10%.

The product mixture will work synergistically due to the fact that the oxidation rate of the free fatty acids will be higher. Reducing the enzymes of the de novo synthesis will have a substantial effect of the availability of triglyceride level and free fatty acids level. Increased transport of fatty acids, increased oxidation of fatty acids and reduced de novo synthesis of fatty acids will reduce the overall plasma level of free fatty acids. A decrease in the level of free fatty acids of at least 2-20% will be obtained already after 30 days compared to the pre-treatment level. The decrease of free fatty acids will have a direct effect on the insuline resistance since lower plasma level of free fatty acids will reduce insuline resistance.

This hypothetical case illustrates that treatment of a subject diagnosed with a condition caused or related to a high level of free fatty acids with the composition of the invention will result in a synergistic metabolic effect causing lowering of the level of free fatty acids which necessarily leads to alleviation of the causes and symptoms of the condition.

### Example 3

### Single and Combined Effects of Niacin, DHA and Carnitine on Parameters of Lipid Metabolism and Insulin Resistance

The aim role of single and combined administration of niacin, DHA and carnitine on insulin sensitivity in Zucker rats as model objects was investigated. Zucker rats are an established model of human early-onset, hyperplastic-hypertrophic obesity and insulin resistance as well as human non-insulin dependent diabetes mellitus.

### Materials and Methods

### Animals

A total of 60 male, 8-10 week old, obese (fa/fa) Zucker rats (Crl:ZUC-Leprfa; Charles River, France) with an initial body weight of 358±24 (mean±SD) g were randomly assigned to five groups of twelve rats each. In addition, 12 male, 8-10 week old, heterozygous lean (fa/+) Zucker rats with an initial body weight of 271±10 g were used as a lean control group. All rats were kept individually in Macrolon cages in a room maintained at 22±1° C. and 50-60% relative humidity with lighting from 06.00 to 18.00 hours. All experimental procedures described followed established guidelines for the care and handling of laboratory animals and were approved by the local Animal Care and Use Committee.

### Diets and Feeding

The rats were fed semi-purified diets which were composed according to the recommendations of the American Institute of Nutrition (AIN)-93G. Five different diets were used differing in their supplementation. The first diet contained no supplement (“lean control”, “obese control”), the second diet 1.5 g DHA (Lonza, Basel, Switzerland) per kg diet (“obese DHA”), the third diet 3 g carnitine (Lonza, Basel, Switzerland) per kg diet (“obese carnitine”), the forth diet 0.75 g niacin (Lonza, Basel, Switzerland) per kg diet (“obese niacin”) and the fifth diet the combination of 1.5 g DHA, 3 g carnitine, and 0.75 g niacin per kg diet (“obese DHA+carnitine+niacin”) (Table 1). Diets were prepared by mixing the dry components and subsequent pelleting with a standard pelleting device (Kahl Laborpressanlage Typ 14-175; Reinbek, Germany). Feed was administered ad libitum, and feed intake was recorded weekly. Experimental diets were fed for 4 weeks. Water was available ad libitum from nipple drinkers during the whole experiment.

TABLE 1
Composition of the experimental diets fed to rats for 4 wk

| | lean and | | | | |
|---|---|---|---|---|---|
| | obese | | | | niacin + DHA + |
| | control | niacin | DHA | carnitine | carnitine |
| | g/kg diet | | | | |
| Corn starch | 530 | 515 | 527 | 529.25 | 511.25 |
| Casein | 200 | 200 | 200 | 200 | 200 |
| Saccharose | 100 | 100 | 100 | 100 | 100 |
| Soybean oil | 70 | 70 | 70 | 70 | 70 |
| Cellulose | 50 | 50 | 50 | 50 | 50 |
| Minerals | 30 | 30 | 30 | 30 | 30 |
| vitamins | 20 | 20 | 20 | 20 | 20 |
| niacin | | 0.75 | | | 0.75 |
| DHA | | | 15 | | 15 |
| carnitine | | | | 3 | 3 |

### Sample Collection

The rats were decapitated under CO2 anesthesia in the fasted state. Whole blood was collected into EDTA polyethylene tubes (Sarstedt, Nürnbrecht, Germany). Plasma was separated from whole blood by centrifugation (1,100×g; 10 min) at 4° C. Lipoproteins [VLDL, LDL and high-density lipoproteins (HDL)] were separated by step-wise ultracentrifugation (900,000×g, 1.5 h, 4° C.; Mikro-Ultrazentrifuge, Sorvall Products, Bad Homburg, Germany). All samples were stored at −20° C. pending analysis.

### Determination of Plasma and Lipoprotein Lipids

Triacylglycerol and cholesterol concentrations in plasma and lipoproteins were determined using enzymatic reagent kits from Biocon (Vöhl-Marienhagen, Germany).

### Determination of Fasting Glucose Concentrations in Plasma

Glucose concentration in plasma was determined by an enzymatic reagent kit from Analyticon Biotechnologies (Lichtenfels, Germany).

### Determination of Non-Esterified Fatty Acids (NEFA) in Plasma

NEFA concentrations in plasma were determined using the NEFA kit from Wako Chemicals (Neuss, Germany).

### Determination of Adipokine Levels in Plasma

Concentrations of leptin in plasma was determined by rat ELISA kits from BioVendor (Heidelberg, Germany). Concentration of high molecular weight adiponectin was determined by a rat ELISA kit from Shibayagi (Ishihara, Shibukawa, Gunma, Japan).

### Results

### Feed Intake, Body Weight Development and Feed Conversion Ratio

Daily feed intake was higher in the obese rats than in the lean rats (P<0.05; Tab. 2). Within the obese rats, daily feed intake was higher in the niacin+DHA+carnitine group than in the carnitine group and the obese control group (P<0.05; Tab. 2). Daily feed intake did not differ between the niacin group, the DHA group, the carnitine group and the obese control group (Tab. 2).

TABLE 2
Feed intake, body weight development and feed conversion ratio of the rats

| | | | | | | obese |
|---|---|---|---|---|---|---|
| | lean | obese | obese | obese | obese | niacin + DHA + |
| | control | control | niacin | DHA | carnitine | carnitine |
| Feed intake (g/d) | 19.7 ± 0.8 ^{c} | 25.2 ± 1.7 ^{b} | 26.2 ± 2.3 ^{ab} | 25.7 ± 2.9 ^{ab} | 25.1 ± 3.4 ^{b} | 27.5 ± 1.9 ^{a} |
| Initial body weight (g) | 271 ± 10 ^{b} | 357 ± 20 ^{a} | 359 ± 29 ^{a} | 359 ± 26 ^{a} | 358 ± 24 ^{a} | 357 ± 21 ^{a} |
| Final body weight (g) | 367 ± 14 ^{b} | 501 ± 23 ^{a} | 500 ± 43 ^{a} | 505 ± 34 ^{a} | 496 ± 31 ^{a} | 502 ± 26 ^{a} |
| Total body weight gain (g) | 96 ± 8 ^{b} | 143 ± 12 ^{a} | 141 ± 19 ^{a} | 146 ± 17 ^{a} | 138 ± 13 ^{a} | 145 ± 9 ^{a} |
| Daily body weight gain (g) | 3.35 ± 0.29 ^{b} | 5.03 ± 0.45 ^{a} | 4.95 ± 0.73 ^{a} | 5.14 ± 0.60 ^{a} | 4.85 ± 0.48 ^{a} | 5.09 ± 0.35 ^{a} |
| Feed conversion ratio (g/g) | 5.90 ± 0.37 ^{a} | 5.02 ± 0.31 ^{c} | 5.35 ± 0.46 ^{b} | 5.01 ± 0.28 ^{c} | 5.18 ± 0.63 ^{bc} | 5.42 ± 0.19 ^{b} |

Values are means ± SD (n = 12 per group). Means with different superscript letters (^{a}, ^{b}, ^{c}, ^{d}) differ (P < 0.05).

Initial and final body weights as well as total and daily body weight gains were higher in the obese rats than in the lean rats (P<0.05; Tab. 2). Within the obese rats, initial and final body weights and total and daily body weight gains did not differ between all groups (Tab. 2). Feed conversion ratio was lower in all obese rats than in the lean rats (P<0.05; Tab. 2). Within the obese rats, feed conversion ratio was higher in the niacin group and in particular in the niacin+DHA+carnitine group than in the obese control group and the DHA group (P<0.05; Tab. 2). Feed conversion ratio in the carnitine group did not differ much from the obese control group (Tab. 2).

### Organ Weights of the Rats

Absolute and relative liver weights were higher in the obese rats than in the lean rats (P<0.05; Tab. 3). Within all obese rats, absolute and relative liver weights did not differ. Relative heart weights were higher in lean rats than in the obese rats (P<0.05; Tab. 3), but did not differ within the obese rats. Absolute heart weights, absolute kidney weights and relative kidney weights did not differ between all groups (Tab. 3).

TABLE 3
Organ weights of the rats

| | | | | | |
|---|---|---|---|---|---|
| | | | | | obese |
| lean | obese | obese | obese | obese | niacin + DHA + |
| control | control | niacin | DHA | carnitine | carnitine |

Absolute organ weights (g)

| | | | | | | |
|---|---|---|---|---|---|---|
| Liver | 11.0 ± 1.2 ^{b} | 23.5 ± 2.8 ^{a} | 24.7 ± 3.7 ^{a} | 24.7 ± 3.7 ^{a} | 25.0 ± 2.8 ^{a} | 24.8 ± 4.3 ^{a} |
| Heart | 1.30 ± 0.12 | 1.36 ± 0.14 | 1.29 ± 0.13 | 1.36 ± 0.13 | 1.36 ± 0.12 | 1.37 ± 0.09 |
| Kidney | 2.33 ± 0.24 | 2.80 ± 0.64 | 2.57 ± 0.49 | 2.59 ± 0.38 | 2.75 ± 0.66 | 2.64 ± 0.43 |

Relative organ weights (mg/g body weight)

| | | | | | | |
|---|---|---|---|---|---|---|
| Liver | 29.9 ± 3.3 ^{b} | 46.9 ± 4.3 ^{a} | 49.4 ± 6.6 ^{a} | 48.6 ± 4.9 ^{a} | 50.4 ± 4.4 ^{a} | 49.2 ± 6.9 ^{a} |
| Heart | 3.54 ± 0.30 ^{a} | 2.71 ± 0.20 ^{b} | 2.59 ± 0.14 ^{b} | 2.70 ± 0.21 ^{b} | 2.73 ± 0.15 ^{b} | 2.73 ± 0.16 ^{b} |
| Kidney | 6.35 ± 0.69 | 5.59 ± 1.28 | 5.16 ± 1.04 | 5.16 ± 0.91 | 5.52 ± 1.17 | 5.27 ± 0.91 |

Values are means ± SD (n = 12 per group). Means with different superscript letters (^{a}, ^{b}, ^{c}, ^{d}) differ (P < 0.05).

### Plasma and Lipoprotein Lipid Concentrations of the Rats

TAG concentrations in plasma and the VLDL+LDL fraction were markedly higher in the obese rats than in the lean rats (P<0.05; Tab. 4). Within the obese rats, TAG concentrations in plasma were lower in the DHA group, the carnitine group, the niacin group and the niacin+DHA+carnitine group than in the control group (P<0.05; Tab. 4). TAG concentration in plasma was lower in the niacin group, the DHA group and the carnitine group than in the niacin+DHA+carnitine group (P<0.05; Tab. 4). The strongest TAG lowering effect was observed in the niacin group which had lower TAG concentrations in plasma than the DHA group and the carnitine group (P<0.05; Tab. 4). With respect to the VLDL+LDL fraction, TAG concentration was only lower in the niacin group than in the obese control group (P<0.05; Tab. 4). TAG concentration in this lipoprotein fraction did not differ between the obese control group, the DHA group and the carnitine group. Surprisingly, TAG concentration in this lipoprotein fraction was higher in the niacin+DHA+carnitine group than in the obese control group, the niacin group, the DHA group and the carnitine group (P<0.05; Tab. 4). Cholesterol concentrations in plasma, VLDL+LDL and HDL were markedly higher in the obese rats than in the lean rats (P<0.05; Tab. 4). Cholesterol in the VLDL+LDL fraction was lower in the niacin group and the niacin+DHA+carnitine group but higher in the DHA group than in the obese control group (P<0.05; Tab. 4). Cholesterol in the VLDL+LDL fraction did not differ between the carnitine group and the obese control group (Tab. 4). Cholesterol in the HDL fraction was significantly lower in the niacin+DHA+carnitine group than in all other obese groups (Tab. 4).

TABLE 4
Plasma and lipoprotein lipid concentrations of the rats

| | | | | | | obese |
|---|---|---|---|---|---|---|
| | lean | obese | obese | obese | obese | niacin + DHA + |
| | control | control | niacin | DHA | carnitine | carnitine |
| Triacylglycerols (mmol/L) | | | | | | |
| Plasma | 1.42 ± 0.20 ^{e} | 6.14 ± 1.10 ^{a} | 3.17 ± 0.40 ^{d} | 4.73 ± 0.98 ^{bc} | 4.41 ± 0.85 ^{c} | 5.21 ± 1.07 ^{b} |
| VLDL + LDL | 0.53 ± 0.08 ^{d} | 2.71 ± 0.23 ^{b} | 2.08 ± 0.40 ^{c} | 2.81 ± 0.52 ^{b} | 2.65 ± 0.57 ^{b} | 3.22 ± 0.33 ^{a} |
| Cholesterol (mmol/L) | | | | | | |
| Plasma | 2.76 ± 0.34 ^{c} | 7.65 ± 0.93 ^{ab} | 6.91 ± 1.13 ^{b} | 8.30 ± 1.00 ^{a} | 7.68 ± 0.92 ^{ab} | 6.87 ± 0.85 ^{b} |
| VLDL + LDL | 1.03 ± 0.14 ^{d} | 2.99 ± 0.32 ^{b} | 2.70 ± 0.27 ^{c} | 3.62 ± 0.53 ^{a} | 3.12 ± 0.39 ^{b} | 2.55 ± 0.26 ^{c} |
| HDL | 2.01 ± 0.23 ^{d} | 4.84 ± 0.42 ^{a} | 4.54 ± 0.54 ^{a} | 4.51 ± 0.45 ^{a} | 4.64 ± 0.50 ^{a} | 4.10 ± 0.56 ^{b} |

Values are means ± SD (n = 12 per group). Means with different superscript letters (^{a}, ^{b}, ^{c}, ^{d}) differ (P < 0.05).

### Concentrations of Glucose, NEFA and Adipokines in Plasma of the Rats

NEFA concentrations in plasma were markedly higher in the obese rats than in the lean rats (P<0.05; Tab. 5). Within the obese rats, NEFA concentrations in plasma were lower in the niacin group, the DHA group, the carnitine group and the niacin+DHA+carnitine group than in the obese control group (P<0.05; Tab. 5). NEFA concentrations in plasma did not differ between rats of the niacin group, the DHA group, the carnitine group and the niacin+DHA+carnitine group (Tab. 5).

TABLE 5
Concentrations of glucose, NEFA and adipokines in plasma of the rats

| | | | | | | obese |
|---|---|---|---|---|---|---|
| | lean | obese | obese | obese | obese | niacin + DHA + |
| | control | control | niacin | DHA | carnitine | carnitine |
| Glucose (mmol/L) | 8.25 ± 0.86 ^{a} | 8.49 ± 0.61 ^{a} | 7.36 ± 0.64 ^{b} | 7.99 ± 0.77 ^{ab} | 7.86 ± 0.75 ^{ab} | 7.79 ± 0.67 ^{ab} |
| NEFA (mmol/L) | 0.73 ± 0.20 ^{c} | 3.53 ± 0.59 ^{a} | 2.25 ± 0.46 ^{b} | 2.42 ± 0.53 ^{b} | 2.41 ± 0.57 ^{b} | 2.33 ± 0.46 ^{b} |
| Leptin (ng/mL) | 2.78 ± 0.04 ^{d} | 5.39 ± 0.68 ^{c} | 6.17 ± 0.71 ^{b} | 7.27 ± 1.09 ^{a} | 6.09 ± 1.29 ^{bc} | 5.63 ± 0.81 ^{bc} |
| Adiponectin (ng/mL) | 140 ± 52 | 119 ± 11 | 134 ± 21 | 120 ± 22 | 122 ± 12 | 125 ± 19 |

Values are means ± SD (n = 12 per group). Means with different superscript letters (^{a}, ^{b}, ^{c}, ^{d}) differ (P < 0.05).

Leptin concentration in plasma was at least 10-fold higher in the obese rats than in the lean rats (P<0.05; Tab. 5). Within the obese rats, leptin concentrations were higher in the DHA group and the niacin group than in the obese control group (P<0.05; Tab. 5). Leptin concentrations in plasma did not differ between the carnitine group, the niacin+DHA+carnitine group and the obese control group (Tab. 5).

Fasting glucose concentration in plasma did not differ between the lean control group and the obese control group (Tab. 5). Within the obese rats, fasting glucose concentration was lower in the niacin group than in the obese control group (P<0.05; Tab. 5). Fasting glucose concentration in the DHA group, the carnitine group, the niacin+DHA+carnitine group and the obese control group did not differ (Tab. 5).

High molecular weight adiponectin concentration in plasma tended to be lower in obese animals than in lean animals (P>0.05). This effect, however, was not significant. Adiponectin concentration in plasma did not differ between the obese groups.

Relative mRNA Concentrations of Genes Involved in Lipogenesis in the Liver of the Rats

Relative mRNA concentrations of the lipogenic transcription factor sterol regulatory element-binding protein (SREBP)-1c and the SREBP-1c target genes fatty acid synthase (FAS) and stearoyl-CoA desaturase (SCD) in the liver were markedly higher in the obese rats than in the lean rats (P<0.05; Tab. 6). Within the obese rats, relative mRNA concentration of SREBP-1c in the liver was lower in the niacin group than in the obese control group (P<0.05; Tab. 6). Relative mRNA concentration of SREBP-1c in the liver did not differ between rats of the DHA group, the carnitine group, the niacin+DHA+carnitine group and the obese control group (Tab. 6). Relative mRNA concentration of FAS in the liver was lower in the carnitine group and the niacin+DHA+carnitine group than in the obese control group (P<0.05; Tab. 6). Relative mRNA concentration of FAS in the liver did not differ between rats of the niacin group, the DHA group and the obese control group (Tab. 6). Relative mRNA concentration of SCD in the liver did not differ between all the obese groups of rats (Tab. 6).

TABLE 6
Relative mRNA concentrations of selected genes involved in lipid metabolism
(lipogenesis, fatty acid uptake, fatty acid oxidation) in the liver of the rats

| | | | | | |
|---|---|---|---|---|---|
| | | | | | obese |
| lean | obese | obese | obese | obese | niacin + DHA + |
| control | control | niacin | DHA | carnitine | carnitine |

Lipogenesis

| | | | | | | |
|---|---|---|---|---|---|---|
| SREBP-1c | 0.16 ± 0.07 ^{c} | 1.00 ± 0.36 ^{a} | 0.66 ± 0.21 ^{b} | 0.81 ± 0.32 ^{ab} | 0.78 ± 0.31 ^{ab} | 0.71 ± 0.21 ^{ab} |
| FAS | 0.02 ± 0.01 ^{c} | 1.00 ± 0.71 ^{a} | 0.70 ± 0.42 ^{ab} | 0.71 ± 0.55 ^{ab} | 0.53 ± 0.49 ^{b} | 0.52 ± 0.36 ^{b} |
| SCD | 0.19 ± 0.10 ^{b} | 1.00 ± 0.37 ^{a} | 1.30 ± 0.44 ^{a} | 1.01 ± 0.45 ^{a} | 0.99 ± 0.33 ^{a} | 0.93 ± 0.37 ^{a} |

Fatty acid uptake

| | | | | | | |
|---|---|---|---|---|---|---|
| FABP | 1.07 ± 0.18 ^{a} | 1.00 ± 0.20 ^{a} | 0.79 ± 0.24 ^{b} | 0.99 ± 0.19 ^{a} | 0.83 ± 0.29 ^{ab} | 1.00 ± 0.20 ^{a} |
| FATP | 0.95 ± 0.51 | 1.00 ± 0.85 | 1.10 ± 0.61 | 1.07 ± 0.90 | 0.93 ± 0.76 | 1.11 ± 0.87 |
| CD36 | 0.41 ± 0.16 ^{b} | 1.00 ± 0.40 ^{a} | 0.93 ± 0.27 ^{a} | 0.91 ± 0.38 ^{a} | 0.87 ± 0.22 ^{a} | 0.79 ± 0.40 ^{a} |
| LPL | 1.21 ± 0.54 | 1.00 ± 0.16 | 1.28 ± 0.51 | 1.26 ± 0.36 | 1.04 ± 0.20 | 1.13 ± 0.41 |

Fatty acid oxidation

| | | | | | | |
|---|---|---|---|---|---|---|
| ACO | 0.79 ± 0.20 | 1.00 ± 0.43 | 1.29 ± 0.44 | 1.27 ± 0.35 | 1.15 ± 0.37 | 1.18 ± 0.59 |
| L-CPT I | 0.71 ± 0.18 | 1.00 ± 0.48 | 1.12 ± 0.50 | 1.31 ± 0.69 | 1.15 ± 0.41 | 0.98 ± 0.50 |

Values are means ± SD (n = 12 per group). Means with different superscript letters (^{a}, ^{b}, ^{c}, ^{d}) differ (P <0.05).

Relative mRNA Concentrations of Genes Involved in Fatty Acid Uptake in the Liver of the Rats

Relative mRNA concentration of the scavenger receptor CD36 in the liver was higher in all obese groups than in the lean group (P<0.05; Tab. 6). Within the obese groups, relative mRNA concentration of CD36 in the liver did not differ.

Relative mRNA concentration of fatty acid binding protein (FABP) in the liver was lower in the niacin group compared to all the other groups (P<0.05; Tab. 6). Relative mRNA concentration of FABP in the liver did not differ between the lean control group, the obese control group, the DHA group, the carnitine group and the niacin+DHA+carnitine group. Relative mRNA concentrations of fatty acid transport protein (FATP) and lipoprotein lipase (LPL) in the liver did not differ between all groups of rats (Tab. 6).

Relative mRNA Concentrations of Genes Involved in Fatty Acid Oxidation in the Liver of the Rats

Relative mRNA concentrations of acyl-CoA oxidase (ACO) and liver-type carnitine-palmitoyltransferase (L-CPT) I in the liver did not differ between all groups of rats (Tab. 6).

### Conclusions

This study was performed to investigate the effect of supplementation of DHA, carnitine or niacin, single or in combination, on the metabolic condition of diabetic rats. Therefore, a study was performed in which 60 male, 8-10 week old, obese (fa/fa) Zucker rats (Crl:ZUC-Leprfa), a well established experimental animal model of type-II diabetes, were fed either a control diet, or the control diet supplemented with 1.5 g DHA (Lonza, Basel, Switzerland) per kg diet (“obese DHA”), 3 g carnitine (Lonza, Basel, Switzerland) per kg diet (“obese carnitine”), 0.75 g niacin (Lonza, Basel, Switzerland) per kg diet (“obese niacin”), or the combination of these three nutrients (1.5 g DHA, 3 g carnitine, and 0.75 g niacin per kg diet, “obese DHA+carnitine+niacin”). As a further group, a non-diabetic lean control group was fed the control diet. The experimental diets were fed for 4 weeks. Compared to the lean, non-diabetic control group, the obese diabetic rats showed a strongly increased feed intake and final body weight, an increased liver weight and strongly increased concentrations of triglycerides, cholesterol and free fatty acids (NEFA) in plasma. Within the groups of obese, diabetic rats, the three nutrients supplemented, individual or in combination, had no effect on feed intake or body weight gains, whereas the feed conversion ratio was increased by niacin as well as by the combination of all three nutrients These three supplements, individual or in combination, had beneficial effects on fasting glucose and NEFA concentrations in plasma. The concentrations of plasma and VLDL/LDL triglycerides were significantly higher for the combination of all three nutrients compared to each individual nutrient suggesting a higher overall turnover rate (release and oxidation) of fatty acids caused by the combination of all three nutrients. Niacin and the combination of all three nutrients had a significant effect on lowering of cholesterol in plasma and VLDL/LDL. The lowering of cholesterol in HDL compared to the obese control was only significant for the combination of all three nutrients but not for the individual nutrients. This leads to the conclusion that the effect of the combination of all three nutrients on lowering of cholesterol is equal (plasma and VLDL/LDL) or even higher (HDL) than the effect of the individual nutrient at a higher overall turnover rate of fatty acids thus significantly improving the metabolic pattern in diabetic subjects. A higher turnover rate of fatty acids is suggesting an increased oxidation rate of fatty acid and reduced synthesis rate of fatty acids leading to a reduction of the ectopic fatty acid deposition in muscle or other tissues leading to an overall better metabolic status of the individual, In order to find an explanation for this on a biochemical base, hepatic genes involved in lipogenesis (fatty acid and triglyceride synthesis), fatty acid oxidation and fatty acid uptake were determined. The fatty acid synthesis in the liver was dramatically increased in the diabetic rats compared to the lean control rats, while there was no significant difference in hepatic fatty acid uptake and oxidation between these two groups of rats. Niacin, DHA, carnitine and the combination of these three nutrients lowered gene expression of fatty acid synthase. The effect in this respect was greatest for carnitine and the combination of the three nutrients. In contrast, the three nutrients, single and in combination, had no significant effect on gene expression of proteins involved in fatty acid uptake and fatty acid oxidation. This leads to the conclusion that fatty acid oxidation was activated by the nutrients rather on the enzyme or receptor (e.g. PPAR-alpha and -gamma) level than on gene expression level. Overall, the study shows that niacin, DHA and carnitine, single and in particular the combination, exert their beneficial effects on metabolic parameters in the diabetic rats.

### Example 4

### Preparation of the Composition

18 g of the components L-carnitine-L-tartrate (LONZA AG, CH-4002 Basel), maltodextrine, natriumcyclamate, natriumcitrate, natriumsaccharine, citronic acid (all from Fluka AG. p. a.), natural lemon flower (Zitronen Aroma 74912-71: Givaudan, CH-8600 Dübendorf), 5 g of DHA (LONZA AG, CH-4002 Basel) and 1 g of niacin (LONZA AG, CH-4002 Basel) are mixed until the mixture is homogenous. Optionally other aroma compounds can be added to the mixture. The mixture is either filled in sachets or filled into capsules or other final galenic forms for oral application. The mixture is packed and is portioned for an amount of L-carnitine related to L-carnitine Base of about 300 to 1500 mg/Sachets

### Example 5

### Preparation of the Composition

18 g of the components L-carnitine-L-tartrate (LONZA AG, CH-4002 Basel), maltodextrine, natriumcyclamate, natriumcitrate, natriumsaccharine, citronic acid (all from Fluka AG. p. a.), natural lemon flower (Zitronen Aroma 74912-71: Givaudan, CH-8600 Dübendorf), 5 g of DHA and 5 g of EPA (both LONZA AG, CH-4002 Basel) and 1 g of niacin (LONZA AG, CH-4002 Basel) are mixed until the mixture is homogenous. Optionally other aroma compounds can be added to the mixture. The mixture is either filled in sachets or filled into capsules or other final galenic forms for oral application. The mixture is packed and is portioned for an amount of L-carnitine related to L-carnitine Base of about 300 to 1500 mg/Sachets.

### Example 6

### Preparation of the Composition

18 g of the components L-carnitine-L-tartrat (LONZA AG, CH-4002 Basel), maltodextrine, natriumcyclamate, natriumcitrate, natriumsaccharine, citronic acid (all from Fluka AG. p. a.), natural lemon flower (Zitronen Aroma 74912-71: Givaudan, CH-8600 Dübendorf), 5 g of DHA and 5 g of EPA (both LONZA AG, CH-4002 Basel) and 5 g of an extract of Spirulina maxima (ALGAEN CORPORATION, NC, USA), are mixed until the mixture is homogenous. Optionally other aroma compounds can be added to the mixture. The mixture is either filled in sachets or filled into capsules or other final galenic forms for oral application. The mixture is packed and is portioned for an amount of L-carnitine related to L-carnitine Base of about 300 to 1500 mg/Sachets.

### Example 7

### Composition for Prevention and Treatment of Cardiovascular Disorders and Dementia

| Compound | mg |
|---|---|
| L-Arginine | 25 |
| Orange flavour | 81 |
| ¹L-Carnitine L-Tartrate | 150 |
| ⁷Folic acid | 0.2 |
| ¹Niacin | 16 |
| ³Vitamine B6 | 1.4 |
| ³Vitamine B12 | 0.0025 |
| ¹DHA from fish oil | 250 |
| Extract of palm oil | 9 |
| ⁹containing tocotrienols | |
| ⁵isoflavone | 250 |
| Lycopene | 6 |

¹LONZA AG, Basel, Switzerland
³QuimDis, LEVALLOIS PERRET CEDEX, FRANCE
⁵PhytoWay Inc, China
⁷Jerafrance, JEUFOSSE, France
⁹Pharmachem laboratories, NJ, USA

### Example 8

### Composition for Prevention and Treatment of Insulin Resistance, Diabetes Mellitus Type 2, Metabolic Syndrome, Dyslipidemias and Obesity

| Compound | mg |
|---|---|
| ²Green tea extracts | 75 |
| ²Mate | 450 |
| ¹L-Carnitin-L-Tartrate | 722.5 |
| ³Fructooligosaccharide | 1000 |
| ³Vitamine B6 | 0.21 |
| ³Vitamine B12 | 0.05 |
| ⁷Folic acid | 0.06 |
| ¹Niacin | 2.4 |
| Biotin | 0.0125 |
| ⁴Vitamine D | 0.0015 |
| ⁶Vitamine E | 1.8 |
| Panthotenic acid | 1.8 |
| ³Mg | |
| ¹DHA 10 % (250 mg/1 g) | 250 |
| L-Arginine | 150 |
| ⁸Glycine | 275 |
| ⁵Soy Protein Powder (10 % Isoflavone) | 125 |

¹LONZA AG, Basel, Switzerland
²Frutarom, Switzerland
³QuimDis, LEVALLOIS PERRET CEDEX, FRANCE
⁴DSM; Switzerland
⁵PhytoWay Inc, , China
⁶Sunvit, Bardowick, Germany
⁷Jerafrance, JEUFOSSE, France
⁸Ferrer, Barcelona, Spain

1. A composition comprising (i) an activator of transport of fatty acids into the mitochondria; (ii) an activator of fatty acid oxidation; and (iii) an inhibitor of fatty acid biosynthesis.

2. The composition according to claim 1, wherein the activator of transport of fatty acids into the mitochondria is L-carnitine, a physiologically acceptable derivative and/or a salt thereof.

3. The composition according to claim 2, wherein L-carnitine, the physiologically acceptable derivative and/or the salt thereof is L-carnitine-tartrate, acetyl-L-carnitine and/or L-carnitine base.

4. The composition according to claim 2, wherein L-carnitine, a physiologically acceptable derivative and/or salt thereof is present in the composition in an amount of 10 to 10000 mg.

5. The composition according to claim 1, wherein the activator of fatty acid oxidation comprises an agent which increases the gene expression and/or activity of PPARα and/or PPARγ in the cells.

6. The composition according to claim 5, wherein the activator of fatty acid oxidation further comprises an agent which increases the AMPK activity, decreases the ACC enzyme activity and/or reduces the allosteric inhibition of malony-CoA.

7. The composition according to claim 6, wherein the agent which increases the AMPK activity, decreases the ACC enzyme activity and/or reduces the allosteric inhibition of malony-CoA is selected from the group consisting of green tea extracts or substances isolated thereof such as EGCG, theaflavine, Bitter melon (Momordia charantia) extract, niacin bound chromium, Chromium Picolinate or sodium acetic acid.

8. The composition of claim 5, wherein the activator of fatty acid oxidation further comprises an agent which increases the gene expression and/or activity of fatty acid oxidizing enzymes.

9. The composition of claim 8, wherein the agent which increases the gene expression and/or activity of fatty acid oxidizing enzymes is selected from the group consisting of peptid YY, genistein, daidzein, fenofibrate, tocopherols, conjugated linoleic acid, fructooligosaccharides, sesamin or the extracted compounds sesamolin or episesamin thereof, C75, cerulenin, carbacyclin, Salacia oblonga root extracts, ureido-fibrate 5, fenofibric acid, megestrol acetate, PUFAs, and resveratrol.

10. The composition of claim 9, wherein the agent which increases the gene expression and/or activity of fatty acid oxidizing enzymes is selected from the group consisting of sesamin or the extracted compounds sesamolin or episesamin thereof, genistein, and resveratrol.

11. The composition according to claim 5, wherein the agent which increases the gene expression and/or activity of PPARα and/or PPARγ is selected from the group consisting of phytanic acid, adiponectin, an agent which increases the endogenous level of adiponectin in the cells, and an agent which decreases the endogenous level of resistin in the cells, Soya isoflavones, lemon polyphenols from lemon peels, phytol contained in fish oil, safflower oil, palm oil, DHA, EPA, arachidonic acid, cinnamon, ethanolamide and microorganism favouring butyrate production in the gut as Lactobacillus gasseri.

12. The composition according to claim 5, wherein the agent which increases the gene expression and/or activity of PPARα and/or PPARγ is selected from the group consisting of phytanic acid, adiponectin, an agent which increases the endogenous level of adiponectin in the cells, and an agent which decreases the endogenous level of resistin in the cells.

13. The composition according to claim 11, wherein the agent which decreases the endogenous level of resistin in the cells is selected from the group consisting of glycine, arachidonic acid and eicosapentaenoic acid.

14. The composition according to claim 11, wherein the agent which increases the endogenous level of adiponectin in the cells is selected from the group consisting of niacin, pantethine, Radix Astragali, astragaloside II, isoastragaloside I, Galegia officinalis, and flaxseed lignane.

15. The composition according to claim 11, wherein the agent which increases the endogenous level of adiponectin in the cells is Radix Astragali and/or Galegia officinalis.

16. The composition according to claim 15, wherein Radix Astragali is present in the composition as extract of Radix Astragali in an amount of 1 to 50000 mg.

17. The composition according to claim 15, wherein Galegia officinalis is present in the composition as extract of Galegia officinalis in an amount of 1 to 50000 mg.

18. The composition according to claim 1, wherein the activator of fatty acid oxidation is selected from the group consisting of soya isoflavones, palm oil or extracts of palm oil containing tocotrienols, DHA and EPA.

19. The composition according to claim 1, wherein the inhibitor of fatty acid biosynthesis is selected from the group consisting of cholic acid, chenodeoxycholic acid, oleic acid, C75, TOFA, FAS, MEDICA, extract of blue algae, Trans10-Cis12-conjugated linoleic acid, tanshinone II, and PUFAs.

20. The composition according to claim 1, wherein the inhibitor of fatty acid biosynthesis is selected from the group consisting of an extract of blue algae, tanshinone II and niacin.

21. The composition according to claim 1, wherein the inhibitor of fatty acid biosynthesis is niacin.

22. The composition according to claim 21, wherein niacin is present in the composition in an amount of 1 to 10000 mg.

23. The composition according to claim 1, wherein the inhibitor of fatty acid biosynthesis is an extract of blue algae or tanshinone II.

24. The composition according to claim 23, wherein the extract of blue algae is an extract of Nostoc commune which is present in the composition in an amount of 1 to 50000 mg.

25. The composition according to claim 1, further comprising an agent which reduces blood glucose.

26. The composition according to claim 25, wherein the agent which reduces blood glucose is selected from the group consisting of carnosol or an extract from rosmarine containing carnosol, curcuma or gurmar and water soluble extracts thereof, ceramides, glabridin, licorice flavonoids, phytosterols, pycnogenol extracted from Pinus pinaster, isoleucine, acarbose, extracts from the root and leaves of C. indica, Korea red ginseng roolets, extracts of fenugreek or Mormordica charantia, extracts of Konja-Mannan and American ginseng and Pterocarpus masupium and Sapogenin extracted from Jamaican bitter yam.

27. The composition according to claim 25, wherein the agent which reduces blood glucose is carnosol or an extract from rosmarine containing carnosol.

28. The composition according to claim 1, further comprising an agent which inhibits lipolysis.

29. The composition according to claim 28, wherein the agent which inhibits lipolysis is niacin or L-arginine.

30. The composition according to claim 1, further comprising an anti-oxidant.

31. The composition according to claim 30, wherein the anti-oxidant is mate tea or mate extract from the leaves of the plant Ilex paraguariensis.

32. The composition according to claim 1 comprising L-carnitine, a physiologically acceptable derivative and/or a salt thereof, DHA and/or EPA and niacin.

33. The composition according to claim 1 comprising L-carnitine, a physiologically acceptable derivative and/or a salt thereof, DHA and/or EPA and an extract of blue algae.

34. The composition according to claim 1 comprising L-carnitine, a physiologically acceptable derivative and/or a salt thereof, DHA and/or EPA and/or soya isoflavones and/or palm oil or an extract of palm oil containing tocotrienols and/or green tea extracts or substances isolated thereof such as EGCG, niacin and L-Arginine.

35. The composition according to claim 1 comprising L-carnitine, a physiologically acceptable derivative and/or a salt thereof, DHA and/or EPA, soya isoflavones, an extract of palm oil containing tocotrienols, niacin and L-Arginine.

36. The composition according to claim 1 comprising L-carnitine, a physiologically acceptable derivative and/or a salt thereof, DHA, soya isoflavones, green tea extracts or substances isolated thereof such as EGCG, niacin, mate tea or mate extract from the leaves of the plant Ilex paraguariensis and L-Arginine.

37. The composition according to claim 1 comprising L-carnitine, a physiologically acceptable derivative and/or a salt thereof, DHA, soya isoflavones, green tea extracts or substances isolated thereof such as EGCG, sesamin or the extracted compounds sesamolin or episesamin thereof, niacin, mate tea or mate extract from the leaves of the plant Ilex paraguariensis, garlic, an extract from rosmarine containing carnosol, and L-Arginine.

38. A pharmaceutical composition comprising the composition according to claim 1 and a pharmaceutically acceptable carrier.

39. A neutraceutical composition comprising the composition according to claim 1.

40. A method for treating insulin resistance, diabetes mellitus type 2, metabolic syndrome, dyslipidemias, obesity, and/or cardiovascular disorders in a subject, the method comprising administering to the subject a therapeutically effective amount of the composition according to claim 1.

41. A method for treating dementia in a subject, the method comprising administering to the subject a therapeutically effective amount of the composition according to claim 1.

42. A method for lowering cholesterol in blood of a subject, the method comprising administering to the subject a therapeutically effective amount of the composition according to claim 1.

43. A method of reducing free fatty acids from mammalian cells comprising administering to a mammal a therapeutically effective amount of the composition according to claim 1.

44. A method of reducing free fatty acid accumulation in mammalian tissues comprising administering to a mammal a therapeutically effective amount of the composition according to claim 1.

45. The method according to claim 40, wherein the subject is a human.

46. The method according to claim 43, wherein the mammalian cells are human muscle cells and/or human liver cells.

47. The composition according to claim 1 for use as a medicament.

48. The composition according to claim 1 for use in a method for treating metabolic syndrome, dyslipidemias, obesity, insulin resistance, diabetes mellitus type 2 and/or cardiovascular disorders in a subject, the method comprising administering to the subject a therapeutically effective amount of said composition.

49. A kit comprising the composition according to claim 1 for the treatment of insulin resistance, diabetes mellitus type 2, metabolic syndrome, dyslipidemias, obesity, dementia and/or cardiovascular disorders.
